(19) United States
(12) Patent Application Publication
(10) Pub. No.: US 2021/0346124 A1
(43) Pub. Date: Nov. 11, 2021

(54) MEDICAL APPARATUS FOR GUIDING A MEDICAL IMPLEMENT AND CONSTRUCTING METHOD THEREOF

(71) Applicant: NORIS MEDICAL LTD., Nesher (IL)

(72) Inventors: Ahron SIEV, Haifa (IL); Rami SIEV, Haifa (IL)

(73) Assignee: NORIS MEDICAL LTD., Nesher (IL)

(21) Appl. No.: 17/380,954

(22) Filed: Jul. 20, 2021

Related U.S. Application Data
(63) Continuation of application No. PCT/IL2020/050001, filed on Jan. 1, 2020.

(30) Foreign Application Priority Data
Jan. 21, 2019 (IL) 264355

(51) Int. Cl. A61C 1/08; A61C 1/00; A61C 3/04

(52) U.S. Cl. CPC A61C 1/082; A61C 3/04; A61C 1/003; A61C 1/0053

(57) ABSTRACT

A medical apparatus for orienting and guiding a medical implement includes a mechanical mechanism that includes a first mechanism portion and a second mechanism portion. The first mechanism portion includes a contact element for disposition on a selected emplacement on a portion of a body, and the second mechanism portion includes an operation module for guidance and support of the medical implement. The mechanical mechanism permits displacement of the contact element and the operation module relative to each other in bidirectional motion of translation along a segment of a straight line, and maintains the medical implement in permanent aim at the selected emplacement. The operation module also supports an implement guide for support an implement drive which includes a motorized drive for operating medical implements coupled thereto.

### TECHNICAL FIELD

Embodiments of the invention relate to medical apparatus and to methods of construction of such medical apparatus, and in particular, for use in dental and orthopedic medicine, such as implantations, including zygomatic implantations, and implants for orthopedic purposes.

### DESCRIPTION OF RELATED ART

Implants and implantations are well known for years, including zygomatic implantations, which are dental implants in the upper jaw, or maxilla.

A problem with zygomatic implantations is that a user, generally a surgeon, has to manually guide and orient a handheld device to drill a bore, without support from imaging equipment and computing facilities, in precise spatial orientation in the three-dimensional volume of a body. The problem is especially acute when a small deviation from the desired orientation of the drilled bore is critical and may cause irreparable damage to the body. The chances of keeping a manually guided drilled bore in aim in the desired direction and orientation are rather precarious. Where precise orientation is crucial, hand-held guidance is inadequate. Straying from a predetermined drill path may result in damage to organ(s) stricken by a drill, which damage is sometimes irreparable. In other words, the problem is how to manually guide and drill a blind bore in precise orientation towards a hidden target point without help from other facilities. Such a problem is encountered, with for example the implantation of a zygomatic intra sinus implant, as shown in FIGS. 26 and 27.

For the sake of illustration and orientation, FIG. 25 shows a portion of a body BDY including bone BNE. A body is meant to be a portion of a creature, including skin, tissue, and bones, such as of humans and of animals. The portion of the bone BNE hidden from sight is shown in dashed lines. A portion of bone BNE may be exposed to view at point A.

One may consider the need to drill a blind bore BBOR starting from an accessible entry point NTRPNT up to but not deeper than a target point TRGPNT. The target point TRGPNT is hidden in the bone BNE which bone may be felt by touch from the exterior BDYEX of the body BDY. A straight axis X crossing through the entry point NTRPNT and the target point TRGPNT exits to the exterior BDYEX of the body BDY at a selected emplacement EMPL.

Problems can be encountered with, for example, the implantation of a zygomatic intra sinus implant, as shown in FIGS. 26 and 27.

FIG. 26 is a front elevation of a face of a patient whereon a trace of the axis X is shown. The axis X marks the direction chosen for drilling a blind bore which starts from an entry NTRPNT disposed at point A on the gingiva (GINGIVA) of the maxilla MAX (see FIG. 27). The angles marked as L and R about the point A as a fulcrum indicate possible angular deviations from the axis X. Care should be taken to avoid deviations from the axis X to mitigate damage to vital organs.

It is imperative to prevent such deviations away from the axis X. For example, a deviation through the angle R is shown to cross the orbital orifice. However, angular deviations on a front elevation are not too difficult to avoid since they may be perceived by extending the direction of the axis of the bore-drilling tool. Rather, the problem lies with deviations in the plane of a cross-section taken through the axis X perpendicular to the front elevation, as shown in FIG. 27.

FIG. 27 is a schematic cross-section of the maxilla MAX showing the desired axis X, the alveolar ridge ALVRG, and the entry point A. The crux of the problem to be solved are the angular deviations γ and δ, away from the axis X, and about the point A as a fulcrum. Unlike the front elevation of the face shown in FIG. 26, the cross-section of FIG. 27 is hidden from view. Tissue can be removed, and an opening can be made through the maxillary sinus wall SNSWL to visually inspect and attempt to orient the drill bore. Nevertheless, such an unnecessary intervention does not avoid mishaps.

For a zygomatic implantation, it is evident that the zygomatic implant has to be anchored in the zygomatic bone Z, shown approximately in dashed lines in FIG. 26. The disposition of the zygomatic bone Z of the skull may be easily detected by a practitioner by palpation of the face with the fingers of a hand, generally below the orbital cavity of the eye. It is next to and opposite the zygomatic bone Z that the practitioner, or user of the medical apparatus, will define the disposition of the selected emplacement EMPL (see FIG. 25). One problem with conventional technology is that it can be difficult to be sure that the desired axis X will pass through the emplacement EMPL and will exit through that defined emplacement.

### SUMMARY

There is presented a mechanism for a medical apparatus to guide a handheld or hand-guided motorized drive to safely drill a blind bore in a desired direction orientation. The medical apparatus is for motorized driving and guiding of medical implements, such as bore drills, and for manually guiding implants, and hand operated tools. The medical apparatus has a mechanical mechanism having a first mechanism portion and a second mechanism portion. The first mechanism portion has a contact element for disposition on a selected emplacement on a portion of a body. The second mechanism portion has an operation module for guidance and support of a medical implement. The mechanical mechanism permits displacement of the contact element and the operation module relative to each other in two directions along a segment of a straight line. Furthermore, the mechanical mechanism permits maintenance of the medical implement in permanent aim at the selected emplacement.

A method is also provided for constructing the medical apparatus for implantation of medical implants, including orthopedic implants, dental implants and zygomatic implants. Moreover, an embodiment is illustrated of the medical apparatus as a structure medical apparatus which is made as one solid and rigid piece of material.

### BRIEF DESCRIPTION OF DRAWINGS

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements, or parts that appear in more than one figure are preferably labeled with a same or similar number in all the figures in which they appear, in which:

FIG. 1 depicts a schematic cross-section through the maxilla,

FIG. 2 shows the medical apparatus, formed as a mechanical mechanism,

FIG. 3 illustrates slide bushings,

FIG. 4 depicts two planes of operation of the mechanical mechanism,

FIGS. 5-7 are alternatives exemplary embodiments of the mechanical mechanism,

FIGS. 8-19 are exemplary embodiments of s contact module and of a contact unit,

FIG. 20 illustrates a stopper element supported by the mechanical mechanism,

FIG. 21 depicts the stopper element,

FIG. 22 illustrates the use of the medical apparatus,

FIG. 23 shows an alternative embodiment

FIG. 24 schematically illustrates embodiments in use, and

FIGS. 25-27 illustrate concepts and problems associated with zygomatic implants.

### DESCRIPTION OF EMBODIMENTS

For the sake of illustration of an exemplary problem, reference is made to dental surgery, for example, to the implantation of an intra sinus zygomatic implant.

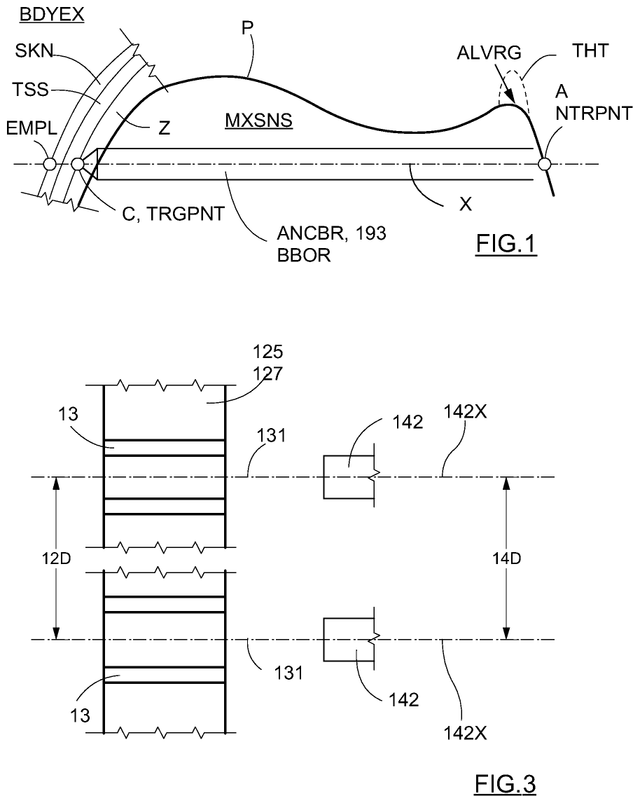

FIG. 1 depicts a schematic cross-section through the maxilla MAX. The cross-section is taken along an axis X of an anchoring bore ANCBR drilled for the implantation of an intra sinus zygomatic implant. The crest of the alveolar ridge ALVRG is void of teeth THT, which nevertheless are shown in dashed lines.

In FIG. 1, on the profile line P, the point A and the point C demarcate the limits of an anchoring bore ANCBR. Both points A and C are disposed on the axis X. The anterior point A marks an entry point NTRPNT of the anchoring bore ANCBR, which entry point is disposed on the alveolar ridge ALVRG. A zygomatic implantation intervention requires typically drilling a blind bore BBOR, usually beginning by drilling at least one preliminary bore followed by an implant anchoring bore ANCBR formed using an anchoring bore drill 193. The blind bore BBOR typically starts from the entry point NTRPNT at the anterior point A, passes through the maxillary sinus MXSNS and ends at the target point TRGPNT, just short of exiting out of the zygomatic bone Z.

In FIG. 1, the posterior point C marks the target point TRGPNT in the zygomatic bone Z, which target point may not be exceeded. Exceeding the target point TRGPNT out of the zygomatic bone Z means piercing out of the face of the patient. Point C is reached after the anchoring bore drill 193 has crossed from the alveolar ridge ALVRG into and through the maxillary sinus MXSNS and has drilled an anchoring bore ANCBR in the zygomatic bone Z. The posterior point C is covered by tissue TSS and skin SKN of the face and is evidently disposed on the axis X of the anchoring bore ANCBR. The axis X passes from the anterior point A via the maxillary sinus MXSNS, crosses the posterior point C, and further continues through the tissue TSS and the skin SKN, and out of the face, thus to the body exterior BDYEX, via the emplacement EMPL selected by the user.

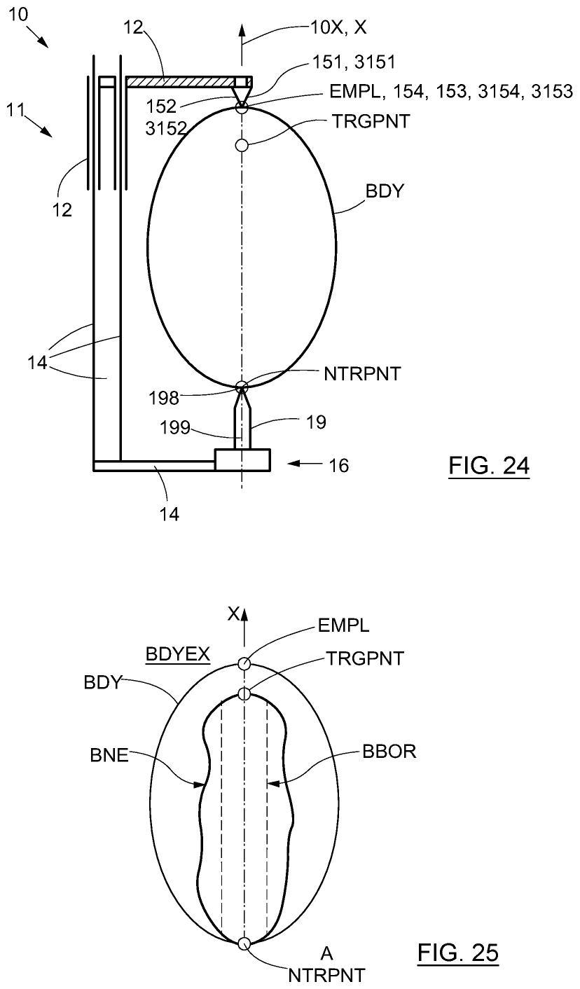

FIG. 24 illustrates an exemplary embodiment of a medical apparatus 10. (Certain reference numerals and characters in FIG. 24 are included for completeness but are described in more detail in other sections of the description below.) The medical apparatus 10 is shown to include a mechanical mechanism 11 having a first portion 12, and a second portion 14. The first portion 12 and the second portion 14 are mutually coupled to permit displacement relative to each other, in limited bidirectional translation motion along a straight segment of a line on the axis 10X. In FIG. 24, the first portion 12 is shown to support the second portion 14 and to allow mutual sliding translation of both portions. Each one of the first portion 12 and the second portion 14 may be said to support the other portion in slidable support.

The first portion 12 supports a contact module 15 which holds a contact element 151, and the second portion 14 supports an operation module 16. The contact element 151 is disposed on the body BDY at the emplacement EMPL. The mechanical mechanism 11 is configured to permanently aim the longitudinal axis 199 of a medical implement 19, for example a bore drill which is supported by the operation module 16, towards the contact element 151. In the description, a bore drill may include preliminary bore drills, final bore drills, implantation bore drills, blind bore drills and the like, for dental and orthopedic implantations and medical interventions in general. By being aimed in orientation towards each other, the medical implement 19 and the contact element 151 are permanently disposed in linear coalignment with and along the aim axis 10X of the medical apparatus 10. Hence, when the contact element 151 is retained on the emplacement EMPL and the medical implement 19 is disposed on the entry point NTRPNT, the former and the latter are connected by the axis 10X. The axis X therefore coincides with the axis 10X in FIG. 24 and both axes pass via the target point TRGPNT. Therefore, when the medical implement 19 is a bore drill driven by the operation module 16, the blind bore BBOR will be aimed at the target point TRGPNT.

It is the user who selects the location of the emplacement EMPL on the skin SKN of the face, to define the orientation of the axis X, which means selecting the orientation of the anchoring bore ANCBR. A straight segment of line stretching from the anterior point A to the selected emplacement point EMPL will thus also support the posterior point C. Thereby, the axis X defines the axis of the anchoring bore ANCBR, and thus also the spatial orientation of the zygomatic implant. That is, once the contact element 151 is disposed on the selected emplacement EMPL, the user will have the certainty that the desired axis X passes through the emplacement EMPL and will exit through that defined emplacement. The use of a dental implement orientation apparatus, or more specifically a dental bore-drill orientation apparatus such as the medical apparatus 10 and/or 310 (described below) provides the certainty of a safe implantation. A user is generally a surgeon, or a medical practitioner such as a dental surgeon or an orthopedic surgeon for example, but the medical apparatus 10, which is also a drill orientation device 10, may also be used by other medical practitioners.

### Mechanical Mechanism

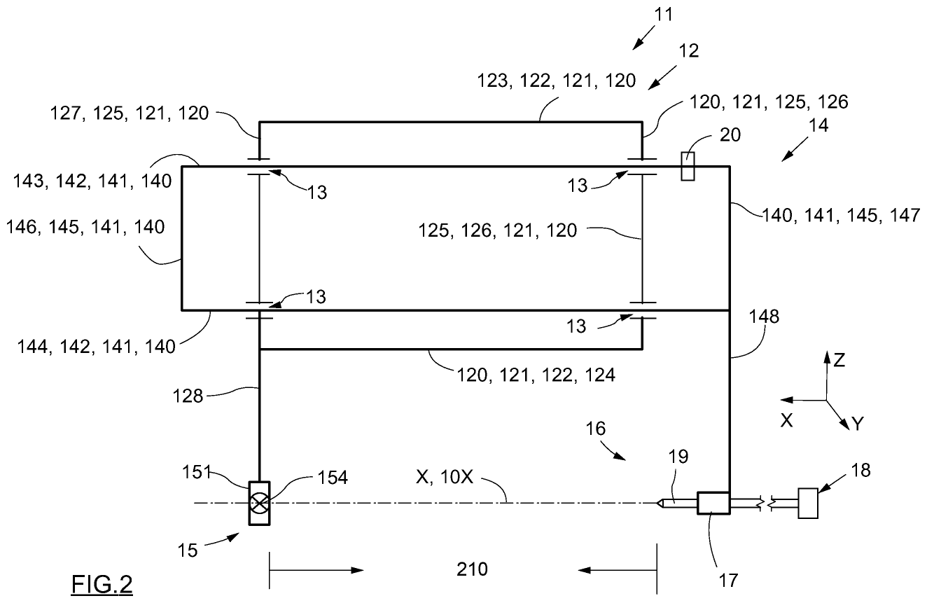

FIG. 2 schematically illustrates an exemplary embodiment of the medical apparatus 10 which is formed as a mechanical mechanism 11 including a first apparatus portion 12 and a second apparatus portion 14, which are mutually coupled for relative motion respective to each other. In FIG. 2, the relative motion is shown as a slidable motion, but alternatively, the relative motion may be other than slidable and the mechanical mechanism 11 may include one or more of bearings, linkages, pivots, linkages, hinges, or a combination thereof.

Each one of both the first and the second portions, respectively, 12 and 14, may be formed as a structure having the general shape of a rectangular frame. A frame may be made of a collection of first frame members 120 forming a boundary for the first portion 12, and a collection of second frame members 140 to form a boundary for the second portion 14. When assembled, the first frame 12 and the second frame 14, may operate in mutually relative bidirectional translation, and as a substantially planar mechanism. Substantially planar means here that the plane is not a geometrical plane but has some thickness since actually, the first frame members 120 and the second frame members 140 are three dimensional. With reference to a set of right-handed Cartesian coordinates shown in FIG. 2, the first portion 12 and the second portion 14 reside in the plane x-z. The first portion 12 and the second portion 14 may translate in the same plane in the positive and in the negative direction of the x-axis.

In the description, up, above, superior, and synonyms thereof refer to the positive values along the z-axis, and down, below, inferior, and synonyms thereof, are related to negative values along the z-axis.

The first portion 12 may have the general shape of a rectangle formed by four first straight members 121 which include at least two parallel first lateral members 122, namely a first superior lateral member 123 and a first inferior lateral member 124.

Perpendicular to the first lateral members 122, the first portion 12 may have two parallel first longitudinal members 125, namely a first minor longitudinal member 126 shorter than a relative thereto longer first major longitudinal member 127. In FIG. 2, the two first lateral members 122 are shown to be parallel to the x-axis of the set of coordinates and the two first longitudinal members 125 are parallel to the z-axis.

The first major longitudinal member 127 has a first additional portion 128 and may thus be longer by that first additional portion than the length of the first minor longitudinal member 126.

As further described hereinbelow, the first frame 12 may include two pairs of laterally disposed linear bearings 13 or bushings 13 (also referred to herein as slide bearings or slide bushings), axially aligned parallel to the x-axis, such as solid sleeve bushings 13 for example. Each one of the longitudinal members 125 may support a couple of linear slide bearings 13 which are disposed thereon in separation apart and parallel to each other and to the x-axis.

Each one slide bearing 13 out of the couple of slide bearings 13 supported on the first longitudinal members 125 is axially co-aligned with a thereto oppositely disposed slide bearing 13. Thus, the couple of slide bearings 13 disposed on the longer first major longitudinal member 127 are coaxially aligned with the couple of slide bearings 13 disposed opposite thereto on the shorter first minor longitudinal member 126. Evidently, the slide bearings 13 are selected to accommodate the second lateral members 142 in sliding fit.

The second portion 14 too may have the general shape of a rectangle formed by four second straight members 141 which include at least two parallel second lateral members 142, namely a second superior lateral member 143 and a second inferior lateral member 144. Perpendicular to the second lateral members 142, the second portion 14 may have two second parallel longitudinal members 145, namely a second minor longitudinal member 146 and a relative thereto longer second major longitudinal member 147.

In FIG. 2, the two second lateral straight members 143 are shown to be parallel to the x-axis of coordinates and the two longitudinal straight members 145 are parallel the z-axis.

Moreover, the second major longitudinal member 147 has a second additional portion 148 and may thus be longer by that second additional portion than the length of the second minor longitudinal member 146.

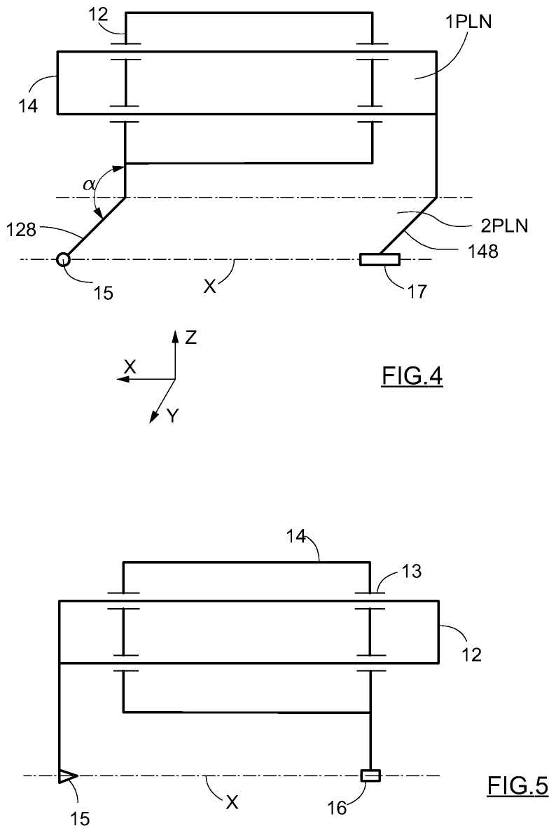

FIG. 3 schematically illustrates two of the slide bushings 13 mounted on the first major longitudinal member 127. With reference to FIG. 2, to couple the second portion 14 to the first portion 12, each one of both second lateral members 142, which have previously been coupled to the second major longitudinal member 147, may be inserted through appropriately mounted and separated apart bushings 13 disposed on both of the first longitudinal members 125. The distance 12D between the axes 131 of the bushings 13 mounted on a longitudinal member 125 is equal to the distance 14D which separates the axes 142X of the two second lateral members 142 which are aligned parallel the z-axis of the set of coordinates shown in FIG. 2. Thereafter, the second minor longitudinal member 146 may be coupled to both second lateral members 142 to form the second portion 14. It is noted that when a stopper element 20 is provided, the stopper element 20 may be mounted, for example, on one of the two second lateral members 142 prior to insertion through the first set of bushings 13.

Once the two second lateral members 142 shown in FIG. 2 exit out of the two bushings 13 supported by the first major longitudinal member 127, the second minor longitudinal member 146, shown in FIG. 2, may be fixedly assembled to the second lateral members 142 to form the second portion 14 into a rigid closed structure of four second straight members 141. Since the bushings 13 are supported in coaxial engagement with the second lateral members 142, the first portion 12 and the second portion 14 will be free to slide in bidirectional translation relative to each other. However, the second portion 14 will remain captive in the first portion 12 and have a limited freedom of translational motion of displacement. In other words, when the second portion 14 of the medical apparatus 10 is slid towards the first portion 12, in the direction of the positive values of the x-axis of the set of coordinates shown in FIG. 2, the second major longitudinal member 147 will be arrested by the first minor longitudinal member 126 of the first portion 12. When the first portion 12 is slid away from the second portion 14, thus in extension of the medical apparatus 10, the second minor longitudinal member 146 of the second portion 14 will arrest the first major longitudinal member 127 of the first portion 12. As described hereinbelow, at least one translation stopper element 20 may be adjustably attached to a second lateral member 142 to precisely restrict the range of translation of the second portion 14 relative to the first portion 12.

FIG. 2 shows that the second additional portion 148, which is a continuation of the second major longitudinal member 147, is disposed parallel to the length of the first additional portion 128 which is a continuation of the first major longitudinal member 127. The first and the second additional portions, respectively 128 and 148, are appropriately disposed in the same plane but mutually opposed to each other, for both motion in translation towards and motion of translation away from each other. In other words, the second additional portion 148 may be slid in translation in a first direction away from the first additional portion 128, to expand and open the medical apparatus 10. To retract and close the medical apparatus 10, the second additional portion 148 is translated in a second direction opposite the first direction.

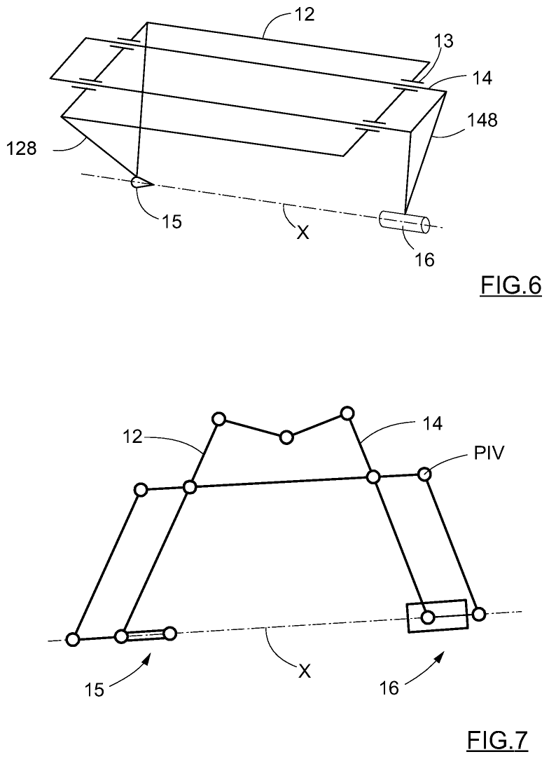

As shown in FIG. 4 such extension or retraction may occur in a first plane 1PL common to the first portion and to the second portion, respectively 12 and 14, and in a second plane 2PL common to the first and the second additional portions, respectively 128 and 148. The second plane 2PL may be disposed at a desired angle α relative to the first plane 1PL, which angle α may be fixed or may be adjustable.

### Alternative Mechanical Mechanisms

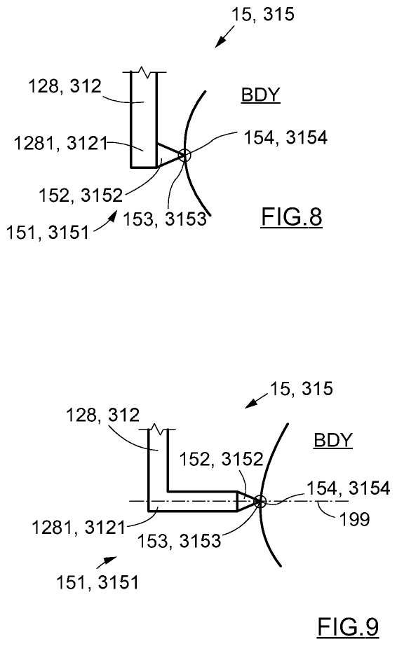

FIGS. 5, 6, and 7, are schematic depictions of alternatives exemplary embodiments out of the many possible embodiments of the mechanical mechanism 11.

FIG. 5 illustrates a mechanical mechanism 11 wherein, relative to FIG. 2, one difference is that the second portion 14 supports the bearings 30. FIG. 6 is different from FIG. 2 in that the first and the second frame members, respectively 120 and 140, are disposed perpendicular and in symmetry relative to the first and second additional portions, respectively 128 and 148. In FIG. 7, the mechanical mechanism 11 is configured as a double planar bar linkages and joints mechanism, wherein the joints have but one degree of freedom of motion. (In FIG. 7, PIV designates a pivot.)

### Contact Module

As shown in FIG. 2, the first additional portion 128 supports the contact module 15 facing the second additional portion 148 which supports the operation module 16. The contact module 15 has a contact element 151 intended to make contact with a portion of the body BDY, such as the face for example. A body BDY is meant to belong to a human or animal, which body BDY may include tissue TSS, skin SKN, and bones BNE, on any one of which the contact element 151 may be disposed. Alternatively, the contact element 151 may be disposed on an auxiliary element 157, shown in FIG. 12, and may be attached to and in contact with the body BDY, for example, by help of an assistant, or by use of a belt-like or a helmet-like device. The contact module 15 and the contact element 151 may be shaped according to the medical case at hand as well as for ease of operation and comfort of manual handling by the user.

Still in FIG. 2, the medical implement 19 supported by the operation module 16 is shown to be coaxially aligned along the axis X in aim towards the contact module 15. The medical implement 19 is thus axially aimed at a contact element 151, or at a contact point 154, or at the emplacement EMPL selected by the user. However, although the medical implement 19 is permanently aimed at the user defined and selected emplacement EMPL, the contact element 151 is not always in direct physical contact with the emplacement EMPL, as described hereinbelow.

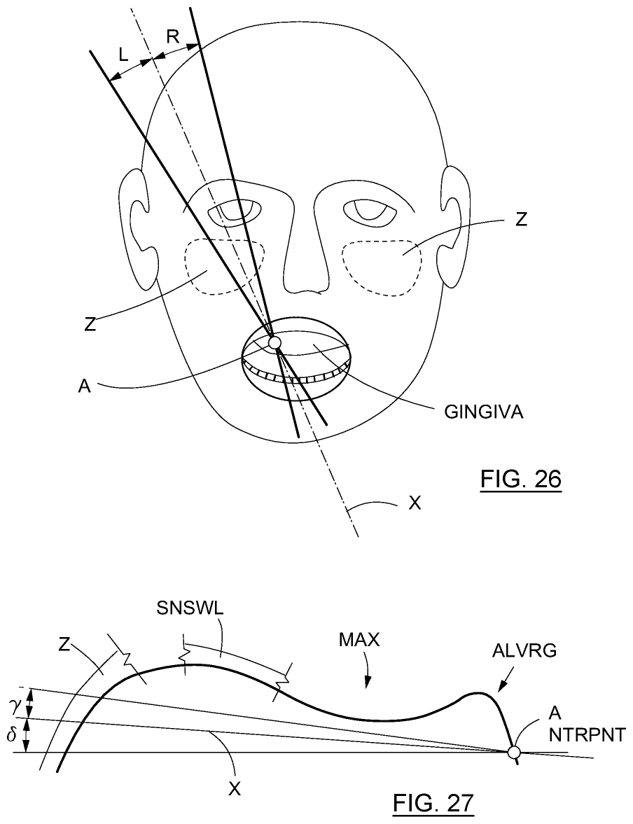

In FIG. 24, the medical implement 19 is depicted in operative disposition on a body BDY. The axis 10X of the medical implement 19 in FIG. 24 coincides with the axis X (e.g., in FIG. 25), which axis stretches from the entry point NTRPNT, via the target point TRGPNT, to the selected emplacement EMPL. The longitudinal axis 199 of the medical implement 19 is aimed at the spike-like contact element 151 which has to be disposed on the selected emplacement EMPL. The tip of the spike 152 is a contact point 154 which is disposed on the axis X, which contact point is common with the selected emplacement EMPL. Thereby, the orientation selected by the user on the straight segment of line from the entry point NTRPNT to the contact point 154 is precisely defined. Such an accuracy of orientation precision allows the user to drill a distribution of closely separated non-intersecting bores extending from various entry points NTRPNT to corresponding selected emplacements EMPL, and moreover, without hazard of intersection of those bores. The user will thus be provided with bore orientation(s) selected as being safe for operation. Safe is meant as being free from causing damage to organs of an anatomy of a body BDY.

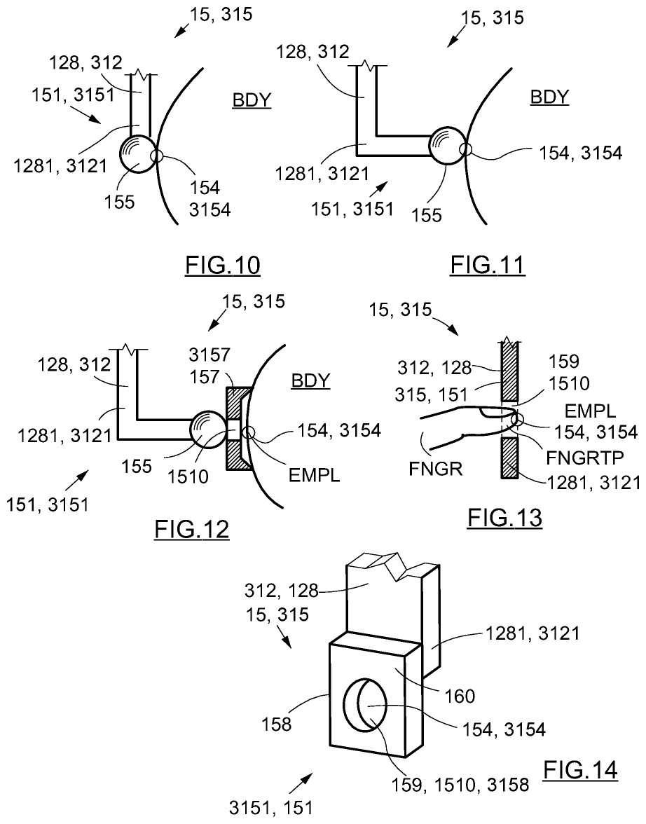

Thus, with the tip 198 of the medical implement 19 on the entry point NTRPNT and the tip 153 (e.g., FIG. 8) of the spike 152 on the selected emplacement EMPL, the medical apparatus 10 is correctly disposed. For example, when properly disposed by the user, the medical apparatus 10 is accurately aimed to precisely guide and orient a medical implement 19 in a direction along which, for example, an implantation bore may be safely drilled.

The contact module 15 may be configured according to the type of surgical intervention, or according to the selected emplacement EMPL, or to fit the auxiliary element 157 to which the contact element 151 will be retained, or as well as with respect to medical considerations. Furthermore, the shape of the contact element 151 may be customized according to request or need. Moreover, ease of handling or retention of the contact element 151 may be enhanced by coupling thereto of a dedicated handling device, which is not shown in the drawings.

Briefly described hereinbelow with reference to FIGS. 8-19 are schematic exemplary illustrations of a few exemplary embodiments of the contact module 15, which may be disposed at the free end portion 1281 of the first additional portion 128.

FIGS. 8 and 9 illustrate a contact element 151, disposed on the free end 1281 of the additional portion 128 or close thereto. The contact element 151 may be configured as a spike 152 which may be appropriate in particular for use of the medical apparatus 10 on a bone BNE. When disposed on a selected emplacement EMPL of a body BDY, the tip 153 of the spike 152 defines the contact point 154 which coincides with both the tip 153 of the spike and the emplacement EMPL.

FIGS. 10 and 11 depict a contact element 151 built as a contact ball 155 which makes contact with the body BDY at a contact point 154. Although not shown in the drawings, instead of a contact ball 155, a contact element 151 configured as a hemisphere may be as effective as the contact ball 155. Likewise, three dimensional bodies of symmetry, like ellipsoids or hemi-ellipsoids may also be used as contact elements 151, although not being shown in the drawings.

FIG. 12 shows an exemplary partial cross-section of an auxiliary element 157 which is disposed between the contact ball 155 and a selected emplacement EMPL at which the axis 199 of a medical implement 19 is aimed. The contact ball 155 is not disposed on and in contact with the selected emplacement EMPL. However, the user may visually recognize and identify the selected emplacement EMPL, which can be optionally marked by the user, via an open aperture 1510 which in the auxiliary element 157. It is up to the user to properly dispose the auxiliary element 157 on the body BDY in continuation of the axis X stretching from the entry point NTRPNT to the selected emplacement EMPL.

It is noted that for a spike 153, the tip 153 of the spike 152 defines the contact point 154 which should be disposed on the by the user selected emplacement EMPL. However, for example, when the contact element 151 is built as a contact ball 155, the contact point 154 of the ball 155 is not limited to one single point on the surface of the contact ball 155. Nevertheless, the physical contact point on the body BDY should be the selected emplacement EMPL.

FIG. 13 depicts an open aperture 1510, or digit window 159, or finger opening 1531 (see FIG. 17) which is opened in the free end portion 1281 of the first additional portion 128. The open aperture 1510 is selected such that when positioned on an emplacement EMPL, a fingertip FNGRTP of a finger FNGR may pass therethrough just to make manual tactile contact with the fingertip FNGRTP as the contact point 154 which coincides with the emplacement EMPL. Before insertion of the fingertip FNGRTP in the open aperture 1510, the open aperture 1510 permits visual inspection and scrutiny of the emplacement EMPL. The emplacement EMPL may have previously indicated by a mark MRK (see FIG. 17).

FIG. 14 depicts the free end portion 1281 of the additional portion 128 to which a removable contact element 158 as the contact element 151 is exemplarily fixedly attached say by releasable mechanical fastening means 160 or fasteners, or by other such means known to those skilled in the art. As true in general for the contact elements 151, the removable contact element 158 may be removable, exchangeable, and replaceable by other different contact elements. Furthermore, the contact element 151 has a digit window 159, thus an open aperture 1510, circular or not, through which a fingertip FNGRTP may pass just enough to touch and palpate the selected emplacement EMPL. The contact point 154, at the center of the digit window 159 or of the open aperture 1510, thus an open aperture 1510 is common with the emplacement EMPL. The longitudinal axis 199 of a medical implement 19 is thus aimed at the selected emplacement EMPL which may be detected and touched through the digit window 159.

FIGS. 15 and 16 show a partial cross-section of a schematic exemplary embodiment of a contact element 151 formed as a gimbal mechanism 1511 having at least three degrees of freedom of rotation for providing enhanced ease of use. FIG. 16 is a partial cross-section of a side elevation cut along a plane A-A shown in FIG. 15. The gimbal mechanism 1511 may be coupled to the free end portion 1281 of the first additional portion 128, or to a thereto attached coupling piece 1515 which is coupled intermediate thereto. Mainly, the gimbal mechanism 1511 may include a core 1512, a rocker 1513, and a finger rest 1514. The core 1512 of the gimbal mechanism 1511 may thus be coupled to the first additional portion 128 or to the intermediary coupling piece 1515.

In FIG. 15, a groove 1521, disposed in the plane x-y of the set of coordinates, is cut either in the first portion 128 or in the intermediary coupling piece 1515. The groove provides a free sliding fit support to the free extremity 1516 of at least three pins 1517 which are fixedly coupled in cantilever to the core 1512. Thereby, the core 1512 is free to rotate about an axis parallel to or coinciding with the axis z of the set of Cartesian coordinates.

In FIGS. 15 and 16, one through pin 1517, fixedly coupled to and passing through the core 1512, supports the rocker 1513. The through pin 1517 is parallel to the axis y of the set of Cartesian coordinates, which axis enters into the paper. The end portions of the through pin 1517 are embedded in the core 1512. As shown in FIG. 16, the mid-portion of the through pin 1517 crosses a core channel 1520. The core channel 1520 separates the core 1512 apart into two core arms 1527 in between of which the rocker 1513 is supported by the through pin 1517. The mid-portion of the through pin 1517 is engaged in and passes in free sliding fit in the curved channel 1518 cut in the rocker 1513. The through pin 1517 is disposed at the center of curvature of the curved channel 1518. Thereby, the rocker 1513 may freely rotate in the clockwise direction CW and in the counterclockwise direction CCW, from one end of the curved channel 1518 to the other end, as shown by arrows marked respectively CW, and CCW. The rocker 1513 may thus pivot over a span of at least 120° in the plane x-z of the set of coordinates.

Two rocker legs 1522 extend away from the curved channel 1518, one rocker leg 1522 from each end of the curved portion 1519 wherein the curved channel 1518 is cut, and parallel to the axis z of the set of Cartesian coordinates. At the free extremity 1523 of the rocker legs 1522 and therebetween, a finger rest 1514 is releasably and interchangeably retained. The finger rest 1514 may be formed as a washer 1524 with an open washer bore 1525 the center of which is the contact point. The washer 1524 is fixedly coupled to two coaxially aligned and diametrically opposite pins 1526. Each one pin 1526 is rotatably supported for rotation by the free extremity 1523 of each one rocker leg 1522. In FIG. 15, the two pins 1526 are shown parallel to the axis x of the set of coordinates. For replacement of the finger rest 1514, the two rocker legs 1522 may be manually extended flexibly away from each other, the finger rest 1514 may be retrieved and may be replaced by another such finger rest 1514. The size of the washer bore 1525 is selected such that when positioned on an emplacement EMPL, a fingertip FNGRTP may pass therethrough just to make tactile contact with the emplacement EMPL. Optionally, the emplacement EMPL may be previously designated by a sign MRK (see FIG. 17). Before insertion of the fingertip FNGRTP in the open washer bore 1525, the finger rest 1514 permits visual inspection and scrutiny of the emplacement EMPL. It is noted that finger rests 1514 may be exchangeable and available in various sizes and shapes to accommodate the user with the intervention at hand.

In FIG. 17, an alternative to the washer-shaped finger rest 1514 is shown as a finger cage 1529 which may substantially envelope the tip FNGRTP of a finger disposed therein. The finger cage 1529 may be fixedly coupled to two coaxially aligned and diametrically opposite pins 1526. Each pin 1526 is rotatably supported for rotation by the free extremity 1523 to each one rocker leg 1522. The tip opening 1530 of the finger cage 1529 which encloses the fingertip FNGRTP may operate in the same manner as the washer bore 1525. The fingertip FNGRTP is entered in the finger cage 1529 via the finger opening 1531 and the center of the tip opening 1530, which can also be considered an open aperture 1510, is the contact point 154.

FIG. 18 depicts a superimposition of a cross-section of the washer-shaped finger rest 1514 and of the finger cage 1529 showing that the fingertip FNGRTP may be considered as a contact point 154 with both the finger rest 1514 and the finger cage 1529. Finger rests 1514 and finger cages 1529 may be selectively interchangeable and have various sizes and configurations to fit the user, the emplacement EMPL, and the type of intervention.

The open washer bore 1525 and the tip opening 1530 of the finger cage 1529 are advantageous for the scrutiny and touch of the selected emplacement EMPL. The user may have indicated the selected emplacement EMPL by having applied an emplacement recognition sign MRK thereon, shown in FIG. 17, such as a cross, a bullet, or a bull's eye for example. Such a sign MRK may be easily detected visually by looking through the open washer bore 1525 or through the tip opening 1530 of the finger cage 1529. It is thereby simple to dispose the center of the washer bore 1525, or of the tip opening 1530, on the sign MRK. An additional advantage relates to the implantation of a zygomatic implant in the zygomatic bone Z. Typically, the layer of tissue TSS and skin SKN that separate the emplacement EMPL from the target point TRGPNT is rather thin. Hence, a fingertip FNGRTP in a finger rest 1514 which is disposed on the emplacement EMPL, may easily sense the approach of an incoming operated motor-driven medical implement 19. Such a tactile detection is possible well before the medical implement 19 pierces out of the zygomatic bone Z. The user will thus be able to stop drilling a blind bore BBOR for example, when the target point TRGPNT is reached, and prevent a blind implantation bore to extend beyond the target point TRGPNT.

For use of the medical apparatus 10, after preliminary medical preparations, the emplacement EMPL and the entry point NTRPNT are selected first, and each one of those may optionally be marked with a sign MRK if desired. Next, the contact point 154 of the contact element 151 is disposed and firmly held on the selected emplacement EMPL, or may be appropriately disposed relative thereto on and in continuation of the axis X extending from the entry point NTRPNT to the emplacement EMPL. Optionally, the contact element 151 may be held in place by an assistant who, alternatively, may hold the auxiliary element 157 if not attached to the body BDY by a belt-like or a helmet-like device. Thereafter, the implement drive 18 is prepared, as described hereinbelow, and is associated with the implement guide 17.

### Operation Module

In FIG. 19, the operation module 16 is shown coupled to the free end portion 1481 of the second additional portion 148 in fixedly attached and accurately oriented direction in aim at the contact element 151. The operation module 16 is dedicated to the support of medical implements 19 and includes mainly an implement guide 17 and an implement drive 18. The implement guide 17 may be coupled to the second additional portion 148, either to an interior side 148a thereof which faces the first additional portion 128 shown in FIG. 2, or to the exterior portion thereof 148b, which faces away from the first additional portion 128. The implement drive 18, which may be configured as an independent module, is associated for operation with the implement guide 17 and is entered in removable support therein from the exterior side 148b of the second additional portion 148.

As shown in FIG. 2, the implement guide 17 is fixedly and permanently aimed in orientation to face the contact module 15. Thereby, a longitudinal axis 199 of a medical implement 19 supported by the implement guide 17 will permanently be coaxially aligned with the axis X, be oriented towards, and aimed at the contact point 154 of the contact element 151.

It is the configuration of the mechanical mechanism 11 which ensures that the longitudinal axis 199 of a medical (or dental) implement 19, supported by the implement guide 17 remains permanently coaligned with the contact point 154. Such coalignment remains true both at standstill and during mutual relative motion between the first portion 12 and the second portion 14 of the mechanical mechanism 11. The medical apparatus 10 ensures that irrespective of the spatial position and attitude thereof and of the operation module 16, once the contact element 151 is disposed on the selected emplacement EMPL, and with the tip 198 of the medical implement 19 on the entry point NTRPNT, the medical implement 19 is aimed at the target point TRGPNT. In other words, the medical implement 19 is aimed at the contact element 151 or contact point 154 which is disposed on the emplacement EMPL, on the straight common linear axis X and 10X which passes through the entry point NTRPNT and the aimed-at target point TRGPNT, as shown in FIG. 24.

FIG. 19 illustrates a schematic embodiment of the operation module 16, showing that the implement guide 17 includes a guide sleeve 172 which may support at least one adaptor sleeve 173. The guide sleeve 172 may be fixedly but releasably and exchangeably coupled to the second additional portion 148, for example by use of a screw thread. The guide sleeve 172 is coupled in coaxial alignment along the axis X which passes through the contact point 154 shown in FIG. 2, and has a larger guide sleeve entrance interior diameter 174 that is followed by a relative thereto smaller guide sleeve interior diameter 175. The adaptor sleeve 173 has an adaptor flange 176 which is configured to be received in the larger guide sleeve entrance interior diameter 174 of the guide sleeve 172. Furthermore, the adaptor sleeve 173 has an adaptor exterior diameter 177 which is configured for guidance and support by the smaller guide sleeve interior diameter 175. Moreover, the adaptor sleeve 173 has an adaptor interior diameter 178 which may be adapted for guidance and support therein of a specific medical implement 19. The guide sleeve 172 may exchangeably receive various adaptor sleeves 173, each one of which may be adapted for guidance and support of a medical implement 19 of specific diameter.

For example, a guide sleeve 172 may have a smaller guide sleeve interior diameter 175 of nominal size of 6 mm and may be fitted with an adaptor sleeve 173 having an adaptor interior diameter 178 made to guide a medical implement 19 of 4.2 mm of diameter. Such a medical implement 19 may be a zygomatic implantation anchoring bore drill 193 or a zygomatic implant, or another type of medical tool. To drill a preliminary bore, the guide sleeve 172 may be fitted with an adaptor sleeve 173 having an adaptor interior diameter 178 matching the diameter of a preliminary bore drill of say, 2.8 mm of diameter. A variety of medical implements 19 may be supported by the operation module 16, to take advantage of the well-defined aim towards the target point TRGPNT, in particular when the target point TRGPNT is hidden from view such as with a zygomatic implant, or for drilling a blind bore BBOR.

FIG. 16 also depicts the implement drive 18 which is operated from the exterior side 148b of the second additional portion 148. The operation device 180 may include a medical implement 19 to be driven by a motor or be operated manually. The operation device 180 may thus include a motorized drive 181, such as a handheld handpiece 182, to which a medical implement 19 is coupled.

For use, the implement guide 17 may have to be adapted to the diameter of the medical (or dental) implement 19 supported by the implement drive 18. However, if appropriate, the nominal interior diameter of the guide sleeve 172 may be used. Next, the adaptor sleeve 173 is introduced via the larger guide sleeve entrance interior diameter 174 of the guide sleeve 172, into the relative thereto smaller guide sleeve interior diameter 175. The introduction of the adaptor sleeve 173 is arrested by the guide sleeve step 179 which is disposed in the interior of the larger guide sleeve entrance interior diameter 174. Finally, the medical implement 19, either coupled to the handpiece 182 or operated manually, is introduced into and out of the adaptor interior diameter 178 or smaller guide sleeve interior diameter 175.

### Stopper Element

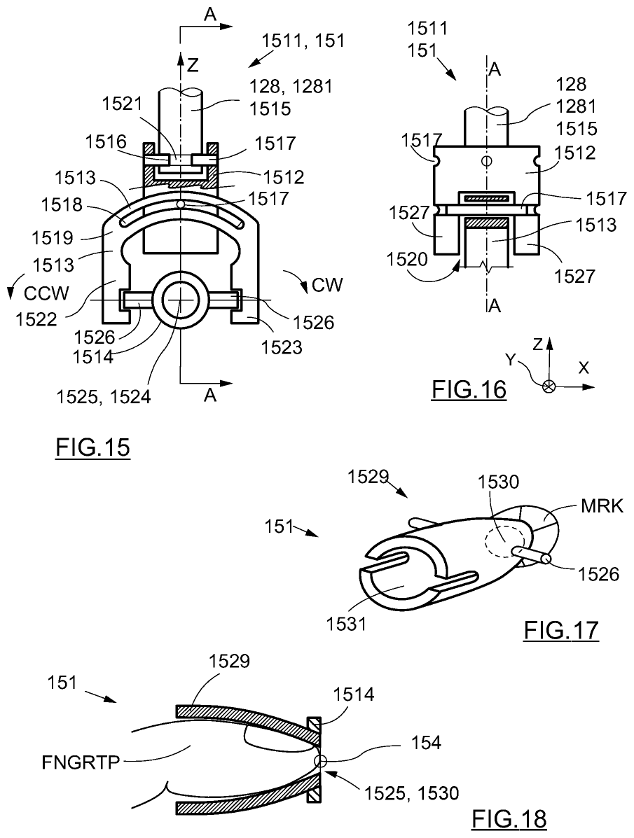

In FIG. 2, a motion stopper element 20 is shown to be disposed on the second superior lateral member 143. The stopper element 20 is intended to arrest motion in one direction by abutment on the first minor longitudinal member 126. Although not shown in the drawings, a motion limiting stopper element 20 may be mounted on the second inferior lateral member 144. Furthermore, a stopper element 20 may be disposed on both of the second lateral members 141. In general, a motion stopper element 20 is arrested by abutment on a first longitudinal member 125. Alternatively, an additional second lateral stopper member 201 may be affixed to the second portion 14 to support a stopper element 20, as shown in FIG. 20. A motion limiting stopper element 20 disposed on a second lateral member 142 or on an additional stopper member 201 may be manually adjustable by coarse adjustment or by fine adjustment. Preferably, the stopper element 20 is adapted for both coarse adjustment and fine adjustment.

FIG. 20 schematically depicts an exemplary embodiment of a mechanical mechanism 11 having an additional stopper member 201 of circular cross section disposed parallel to the first and second lateral members, respectively 122 and 142. Preferably, the stopper member 201 is a screw threaded rod. One end of the stopper member 201 is coupled to the second minor longitudinal member 146 and the other end is coupled to the second major longitudinal member 147. Passages 202 opened in both of the first longitudinal members 125 allow free travel therethrough of the stopper member 201. The stopper element 20 is shown to be supported on the stopper member 201, between the first minor longitudinal member 126 and the second major longitudinal member 147.

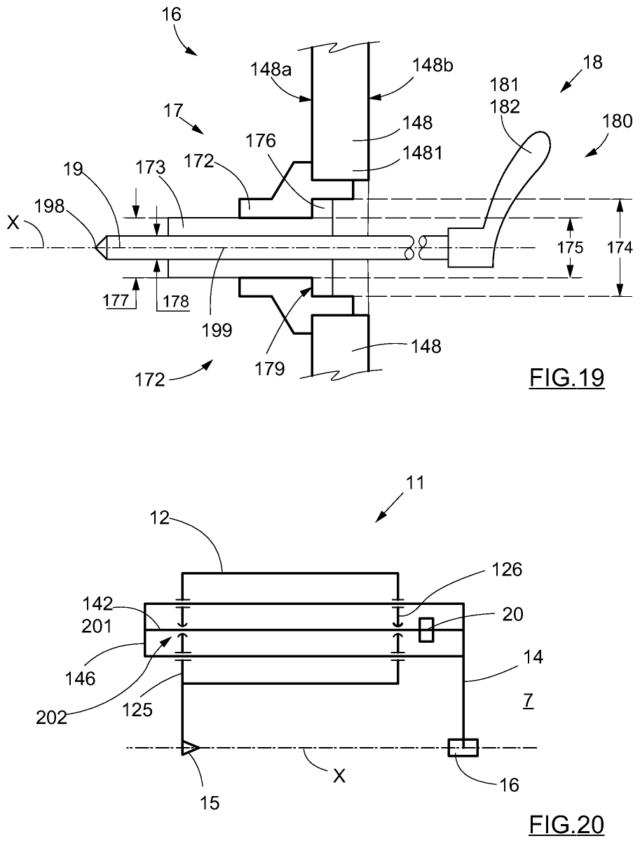

FIG. 21 is a schematic illustration of a partial cross section of an exemplary embodiment of an adjustable motion-limiting-stopper element 20, or stopper 20 for short. The stopper 20 is accommodated to arrest motion of the second portion 14 relative to the first portion 12, thus of the operation module 16 towards the contact module 15, and vice versa. The stopper 20 allows the user to select a desired longitudinal distance of separation 210, or gap of separation 210, shown in FIG. 2, between the tip 198 of the medical implement 19 and the contact element 151, by precise adjustment of the disposition of the stopper 20. The selected longitudinal distance 210 may thus be set as a limit beyond which mutual relative motion between the operation module 16 and the contact module 15 cannot be shortened. For example, with the contact module 15 firmly retained on the emplacement EMPL, and with the tip 198 of a medical tool 19, such as for example a medical bore drill positioned on an entry point NTRPNT, the selected motion-limiting-position of the stopper 20 allows the user to drill a blind bore BBOR up to the user selected bore depth limit, but not further on, or deeper. By proper adjustment of the motion-limiting-stopper 20, the user is thus able to safely drill a blind bore BBOR precisely to a predetermined depth, which drill depth may be limited to not trespass the target point TRGPNT.

In FIG. 21, the stopper 20 is shown to be disposed for example on a screw threaded lateral stopper member 201 having a fine-lead screw thread. A spring-loaded piston 203 supports a blade 204 which is urged between screw threads of the stopper member 201 by a resilient element 205. The resilient element 205, such as a helical spring 205 for example, has a first end which abuts against a bottom portion of the piston 203. A second end of the resilient element 205 abuts against the interior bottom of a cup 206.

The cup 206 “hangs” on the lateral stopper member 201 which passes therethrough via two diametrically opposite cup openings 211 that are provided therein. The cup opening slots 211 may be configured as longitudinal slots configured for free passage therein of the stopper member 201. A plunger 209, which supports a button 207, is mounted on the piston 203. When the button 207 is depressed to force the plunger 209 and next the piston 203 against the resilient element 205, the two diametrically opposite plunger slots 213 may be aligned with the two diametrically opposite cup openings 211 for the lateral stopper member 201 to pass through both cup and plunger slots, respectively 211 and 213.

With the blade 204 in engagement in the screw threaded stopper member 201, it is easy to manually rotate the stopper element 20 about the stopper member 201. For each complete turn of rotation of the stopper element 20, the blade 204 is displaced by a distance equal to the lead of the screw thread of the lateral stopper member 201. For precise and fine adjustment of the stopper element 20, a screw thread having a fine lead of 0.5 mm to 1 mm may be advantageous.

To displace the stopper element 20 in coarse adjustment by translation along the lateral stopper member 201, which may be faster than by rotation for fine adjustment, the button 207 is pushed to depress the plunger 209 and the piston 203, which when depressed, retrieves the blade 204 out of the threads of the screw thread stopper member 201. The two diametrically opposite open plunger slots 213 provided in the plunger 209 permit passage therethrough of the lateral stopper member 201, and further, permit the displacement of the plunger 209 perpendicular to and towards the lateral stopper member 201. Hence, when the plunger 209 is depressed, the plunger 209 pushes the piston 203 which in turn, compresses the spring 205. Thereby, the blade 204 is extracted out of the screw threads of the stopper member 201, and the stopper element 20 may be translated along the stopper member 201 bidirectionally for coarse position adjustment.

Advantageously, the stopper element 20 permits both: precise adjustment of the selected longitudinal distance 210 by rotation about the stopper member 201, as well as coarse adjustment and faster displacement in translation following the depression of the plunger 209 and sliding translation along the stopper member 201.

For assembly of the stopper element 20, the resilient element or spring 205 is introduced in the cup 206 with the piston 203 thereon and with the blade 204 disposed away from the spring 205. Next, the plunger 209 is mounted on the piston 203. In turn, by pushing the button 207, the plunger 209 is depressed against the spring-loaded piston 203. If necessary, the button 207 may be rotated to expose the two diametrically opposite plunger slots 213 in alignment with the two diametrically opposite cup slots 211. Then, the stopper member 201 is passed through the aligned plunger openings 213 and cup openings 211, and next, the button 207 is released for the blade 204 to engage the screw thread of the stopper member 201.

### Use of the Medical Apparatus 10

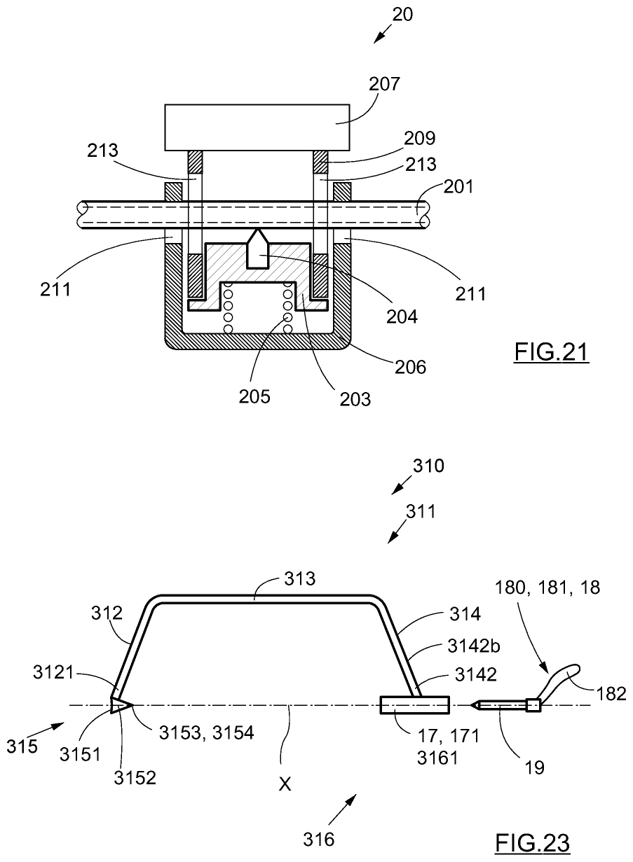

Zygomatic implants are used for dental rehabilitation when there is insufficient bone BNE in the upper jaw. The medical apparatus 10 may be used for both intra maxillary and extra-maxillary zygomatic implantations. An example, simplified for ease of description of an intra maxillary sinus implantation, and for use of the medical apparatus 10 in comparison of a guided and of an unguided conventional implantation is described hereinbelow with reference to FIGS. 1 and 22.

FIG. 22 provides a simplified and brief illustration of the exemplary use of the medical apparatus 10 for an intra maxillary sinus zygomatic implantation with reference to FIG. 1. First, the second portion 14 of the medical apparatus 10 is extended away from the first portion 12. Then, the operation module 16 is formed by joining thereto of the implement drive 18 that supports a dental implement 19 which is coupled to and is driven by a handpiece 182. An adaptor sleeve 173 may be used. The position of the stopper element 20 is adjusted. The contact piece 151 of the contact module 15 is disposed on the face of a patient, on the skin SKN opposite the zygomatic bone Z, on which the user has selected the emplacement EMPL by manual palpation. With the contact piece 151 on the emplacement EMPL and the tip 198 of the dental implement 19 on the entry point NTRPNT shown in FIG. 1, the handpiece 182 is operated to drill a blind bore. A blind bore BBOR is drilled until arrested by the stopper 20 and/or by a finger-palpated detection of the incoming drill. Then, the handpiece 182 is retrieved out of the implement guide 17. Finally, a handheld insertion and anchoring dental implement 19 may be used, optionally via the implement guide 17, to terminate the implantation.

It is noted that use of the medical apparatus 10 averts the guesswork regarding free handheld unguided direction of orientation and depth of bore drilling.

### Mechanical Structure of an Additional Embodiment

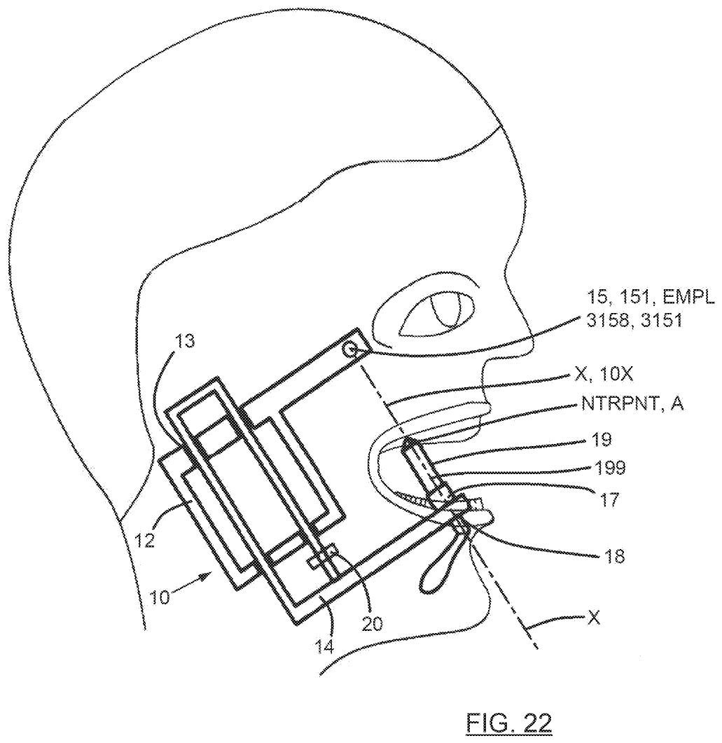

FIG. 23 schematically illustrates an additional exemplary embodiment of the medical apparatus as a structure medical apparatus 310 which is formed as a solid state mechanical structure 311. In contrast with the mechanical mechanism 11 described hereinabove, the mechanical structure 311 may be made as one rigid and robust unitary, thus single piece of material, including a contact unit 315 and a guidance unit 316. The mechanical structure 311 includes a first structure portion 312 and a second structure portion 314, which are mutually rigidly coupled together by a third structure portion 313 to form a rigid, generally arcuate shape.

The structure medical apparatus 310 may be produced from medical practice compliant materials which may include metals, plastics, and synthetic materials. Fabrication of the structure medical apparatus 310 may be achieved by conventional manufacturing processes including additive printing manufacture, which processes are well known to those skilled in the art. The structure medical apparatus 310 may be made in different sizes and shapes from which one structure apparatus 310 of appropriate size and shape may be selected for a specific medical procedure.

In the description, up, above, superior, and synonyms thereof refer to the positive values along the z-axis, and down, below, inferior, and synonyms thereof, are related to negative values along the z-axis.

As shown in FIG. 23 the first structure portion 312 supports a contact unit 315 facing the guidance unit 316 which is supported by the second structure portion 314. The contact unit 315 may be identical or similar to one of the embodiments of the contact module 15 described hereinabove with reference to FIGS. 8 to 14 for the mechanical mechanism 11. For example, the contact unit 315 may include a spike-like contact element 3151 including a spike 3152 with a tip 3153 as a contact point 3154. The contact element 3151 of the contact unit 315 may be provided for making contact with an exterior portion of the body BDY, such as the face for example. A body BDY is meant to belong to a creature, which body BDY may include tissue TSS, skin SKN, and bones BNE, on each one of which the contact element 3151, may be disposed. Alternatively, the contact element 3151 may be disposed on an auxiliary element 157 (or 3157), shown in FIG. 12. The auxiliary element 157 may be held in contact with the body BDY, such as for example, by help of an assistant, or by a belt-like or a helmet-like device, but are not shown in the drawings. The contact unit 315 and the contact element 3151 may be shaped according to needs of the medical case at hand as well as for ease of operation and comfort of manual handling by the user. If desired, a contact element 3151, identical or similar to one of the embodiments of the contact element 151 of the contact module 15 described hereinabove with reference to FIGS. 14 to 18 for the mechanical mechanism 11, may be used on the first extremity 3121 of the first structure portion 312.

Still in FIG. 23, the guidance unit 316 for support and guidance of the medical (or dental) implements 19 includes a guidance tube 3161 shown to be coaxially aligned along the axis X in aim towards the contact unit 315. Medical implements 19 are thus axially aimed at a contact element 3151, or contact point 3154, or emplacement EMPL selected by the user for disposition thereon of the contact unit 315. However, although the medical implement 19 is permanently aimed at the by the user defined and selected emplacement EMPL, the contact element 151 is not always in direct physical contact with the emplacement EMPL, as described hereinbelow.

In FIG. 24, the medical implement 19 is depicted in operative disposition on a body BDY. The longitudinal axis 199 of the medical implement 19 coincides with the axis X (e.g., FIG. 25) which stretches from the entry point NTRPNT, via the target point TRGPNT, to the selected emplacement EMPL. The longitudinal axis 199 of the medical implement 19 is aimed at the spike-like contact element 151 or 3151, which is disposed on the selected emplacement EMPL. The tip 153 or 3153 of the spike 152 or 3152, is a contact point 154 or 3154, which is disposed on the axis X. Thereby, the direction of orientation selected by the user on the straight segment of line stretching from the entry point NTRPNT to the contact point 154 or 3154 is precisely defined. Such an accuracy of orientation precision allows the user to drill a distribution of closely separated apart non-intersecting bores. The user will thus be provided with bore orientation(s) selected as being safe for operation. Safe is meant as being free from causing damage to organs of an anatomy of a body BDY. Thus, with the tip 198 of the medical implement 19 on the entry point NTRPNT and the tip 153 or 3153 of the spike 152 or 3152 on the selected emplacement EMPL, the medical apparatus 10 is properly disposed. For example, when properly disposed by the user, the medical apparatus 10 and the structure medical apparatus 310 are accurately aimed to precisely guide and orient a medical implement 19 in a direction along which a blind bore BBOR, for example an implantation bore, may be safely drilled.

The contact unit 315 may be configured according to the type of surgical intervention and/or according to the selected emplacement EMPL on a portion of the body BDY to which the contact element 151 or 3151 will be retained, or to the auxiliary element 157 or 3157. Furthermore, the shape of the contact element 151 or 3151 may be customized according to request or need. Moreover, ease of handling or retention of the contact element 3151 may be enhanced by coupling thereto of a dedicated device for ease of handling, which is not shown in the drawings.

Briefly described hereinbelow with reference to FIGS. 8-19 are schematic illustrations of a few exemplary embodiments of the contact unit 315, which may be disposed on the first structure portion 312.

FIGS. 8 and 9 illustrate a contact element 151, or 3151, configured as a spike 152, or 3152, which may be appropriate in particular for use of the medical apparatus 10 on a bone BNE. When disposed on a selected emplacement EMPL of a body BDY, the tip 153, or 3153, of the spike 152, or 3152, defines the contact point 154, or 3154, which coincides with the spike tip 153, or 3153.

FIGS. 10 and 11 depict a contact element 151, or 3151, built as a contact ball 155 which makes contact with the body BDY at a contact point 154 or 3154. A contact element 151, or 3151, terminated as a hemisphere may be as effective as a contact ball 155.

FIG. 12 shows an exemplary partial cross-section of an auxiliary element 157 which is disposed intermediate the contact ball 155 and a selected emplacement EMPL at which the longitudinal axis 199 of a medical implement 19 is aimed. The contact element 151 or 3151 with the contact ball 155, is not disposed on the selected emplacement EMPL. However, the user may visually recognize and identify the selected emplacement EMPL he defined via an open aperture 1510 entered in the auxiliary element 157, for disposition thereof on the segment of straight line stretching from the entry point NTRPNT to the target point TRGPNT, as shown in FIG. 24.

FIGS. 13 and 14 depict the free end portion 1281 of the first additional portion 128, or the first extremity 3121 of the first structure portion 312, into which a contact element 151, or 3151, is formed, or is exemplarily fixedly attached say by releasable mechanical fasteners 160, or by other affixation means known to those skilled in the art. The contact element 3151 or 151 may be removable, exchangeable, and replaceable by other different contact elements. Furthermore, the contact element 151 or 3151, may have a digit window 159, thus an open aperture 1510 or 3158, through which a fingertip FNGRTP may pass just enough to touch and palp the selected emplacement EMPL. The center of the digit window 159, or of the open aperture 3158, may be considered as being the contact point 3154 or 154. The longitudinal axis 199 of a medical implement 19 is aimed at the selected, through the digit window 159, or 3158, detected, emplacement EMPL.

The contact element 3151 may also be configured as a gimbal mechanism 1511 for attachment to the first structure portion 312, according to description related to FIGS. 15 to 18 hereinabove.

In FIG. 23, the structural implement guide 171 of the mechanical structure 311 is shown coupled to the second extremity 3142 of the second structure portion 314, in fixedly attached and accurately oriented direction. The structural implement guide 171 is dedicated to the support of medical implements 19 and is configured to support implement drive 18. The structural implement guide 171 is fixedly coupled to the extremity 3142.

The implement drive 18 may be associated for operation with the structural implement guide 171 and is entered therein from the exterior side 3142b of the second structure portion 314. The operation device 180 may include a medical implement 19 to be driven a motor or be operated manually. The operation device 180 may thus include a motorized drive 181, such as a handheld handpiece 182, to which a medical implement 19 is coupled. In contrast with the mechanical mechanism 11, the medical implement 19 supported by the mechanical structure 311 is translated solely by the motion imparted to the operation device 180 relative to a structural guide sleeve of the structural implement guide 171. This is different from the simultaneous translation of both the implement guide 17 together with the implement drive 18 as possible with the mechanical mechanism 11.

It is the configuration of the mechanical structure 311 which ensures that the longitudinal axis 199 of a medical implement 19, such as an orthopedic implement or a dental implement supported by the structural implement guide 171, remains permanently coaligned with the contact element 151 or 3151, or contact point 154 or 3154. The structure medical apparatus 310 ensures that irrespective of the spatial position and attitude thereof, once the contact element 151 or 3151 is disposed on the selected emplacement EMPL, and with the tip 198 of the medical implement 19 on the entry point NTRPNT, the medical implement 19 is aimed at the target point TRGPNT. In other words, the medical implement 19 is aimed at the contact point 154 which is disposed on the emplacement EMPL, on the straight linear axis X or 10X which passes through the entry point NTRPNT and the aimed-at target point TRGPNT, as shown in FIG. 24.

FIG. 23 illustrates a schematic embodiment of the structural implement guide 171 which is formed as a structural guide sleeve which is fixedly coupled to the second portion extremity 3142 of second portion structure 314. The structural guide sleeve is coupled in coaxial alignment along the axis X which passes through the contact point 154 or 3154, as shown in, respectively, FIGS. 2 and 23. The structural guide sleeve may be configured as a guidance tube 3161 tube having a smaller interior diameter 175 which may be accepted as a nominal interior diameter. The smaller interior diameter 175 may be adapted to receive therein and support adaptor sleeves 173 or the guidance tube 3161 may have a smaller interior diameter 178 which is adapted for guidance and support therein of a specific medical implement 19.

Like the guide sleeve 172 of the mechanical mechanism 11, the structural guide sleeve may have a smaller guide sleeve interior diameter 175 which may have a nominal diameter size of 6 mm for example, and may be fitted with an adaptor sleeve 173 having an adaptor interior diameter 178 fitted to guide a medical implement 19 of 4.2 mm of diameter. Such a medical implement 19 may be a zygomatic implantation anchoring bore drill 193, or a medical implant such as a zygomatic implant, or another type of medical tool. To drill a preliminary bore, the guide sleeve 172 may be fitted with an adaptor sleeve 173 having an adaptor interior diameter matching the diameter of a preliminary bore drill of say, 2.8 mm of diameter. Various sizes of adaptor sleeve 173 may be provided to match and support a plurality of medical implements 19. A variety of medical implements 19 may be supported by the structural implement guide 171, to take advantage of the well-defined aim towards the target point TRGPNT, in particular when the target point TRGPNT is hidden from view such as with a zygomatic implant or when drilling a blind bore BBOR for other purposes.

Alternatively, the structural guide sleeve may have a smaller guide sleeve interior diameter 175 adapted to fit a specific medical implement 19.

### Use of the Structure Medical Apparatus 310

The structure medical apparatus 310 is another embodiment of the medical apparatus 10, but both provide guidance to ensure correct implantation of implants in desired and selected directional orientation. Use of the structure medical apparatus 310 is similar to the use of the medical apparatus 10, taking into consideration that the structure medical apparatus 310 is made as one single piece, contrary to the first portion 12 and the second portion 14 of the medical apparatus 10.

First, since it is not extensible and retractable, a structure medical apparatus 310 of shape and size appropriate for the intervention at hand has to be selected out of a set of such apparatus. Then, the operation module 16 is formed by joining thereto of the implement drive 18 that supports a medical (or dental) implement 19 which is coupled to and is driven by a handpiece 182. An adaptor sleeve 173 may be used. The contact piece 3151 of the contact unit 315 is disposed on the face of a patient, on the skin SKN opposite the zygomatic bone Z, on which the user has selected the emplacement EMPL by manual palpation. The dental implement 19 is introduced ready to extend out of the structural guide sleeve of the structural implement guide 171.

With the contact piece 3151 on the emplacement EMPL and the tip 198 of the dental implement 19 having been pushed onto the entry point NTRPNT shown in FIG. 1, the handpiece 182 is operated to drill a blind bore. A blind bore BBOR is drilled until arrested by a finger-palpated detection of the incoming drill. Then, the handpiece 182 is retrieved out of the structural implement guide 171. Finally, a handheld insertion and anchoring dental implement 19 may be used, optionally via the structural implement guide 171, to terminate the implantation to terminate the implantation.

It is noted that use the structure medical apparatus 310 averts the guesswork regarding free handheld unguided direction of orientation and depth of bore drilling.

There has thus been described a medical apparatus 10 and a method for guiding a medical implement 19. The medical apparatus 10 is generally intended for use by surgeons or medical practitioners like dental surgeons but is not limited to the field of medicine. Medical implement 19 is considered to include medical tools and medical items used for the performance of a medical intervention or of medical procedures. Such tools may include bore drills, medical implants and osteotomic tools, either motor driven or handheld.

The medical apparatus 10 may have a mechanical mechanism 11 with a first mechanism portion 12 that includes a contact module 15 supporting a contact element 151, which may present a specific contact point 154, such as the tip of a spike, or have a point of a surface that may make contact with a portion of a body BDY. Body BDY refers to the body of a living vertebrate creature and includes bone BNE, skin SKN, and tissue TSS. The location for the contact element 151 to make contact with the body BDY is defined by the user of the medical apparatus 10 as a selected emplacement EMPL.

The mechanical mechanism 11 may also have a second mechanism portion 14 that includes an operation module 16 which is configured for guidance and support of the medical implement 19. Guidance means maintenance in a desired direction of orientation. In other words, prevention of the straying of the medical implement 19 in a direction of orientation other than the desired direction, which straying may be due to, for example, encountered obstacles that cause deviation from the desired direction.

The mechanical mechanism 11 is further configured to permit displacement of the contact element 151 and of the operation module 16 relative to each other. One out of both of the couple including the contact element 151 and the operation module 16 may be kept at standstill while the other one out of the couple may move closer thereto or away therefrom. The relative movement may thus be bidirectional but is limited to motion of translation along a limited segment of a straight line. Furthermore, the mechanical mechanism 11 maintains the medical implement 19 in constant and permanent aim at and towards the user selected emplacement EMPL. This thus also means that the mechanical mechanism 11 maintains the medical implement 19 in constant and permanent aim at the contact element 151 which is disposed on the selected emplacement EMPL.

The operation module 16 is further configured to support a plurality of various medical implements 19, and to support an implement guide 17 as well as an implement guide 17 associated for operation with an implement drive 18. The implement guide 17 may support a guide sleeve 172 for guidance of a medical implement 19, wherein the guide sleeve 172 is configured to support one or more adaptor sleeves 173.

The implement drive 18 is configured to support a motorized drive 181 such as a hand-held power tool 182 to which a medical tool 19 may be coupled, and to drive the medical implement 19 into motion. The implement drive 18 is independent from the medical apparatus 10 and from the mechanical mechanism 11. Therefore, the implement guide 17, which is fixedly coupled to the mechanical mechanism 11, is open to receive and guide a handheld medical tool 19 for manual operation thereof, either before or after associating the implement drive 18 for operation with the implement guide 17. In operation, the motorized drive 181 and the implement guide 17 may be maintained jointly together by manual retention.

The contact element 151 supported by the contact module 15 may have an open aperture 1510 which is configured for inspection therethrough by visual scrutiny and/or by manual tactile palpation of the selected emplacement EMPL. The selected emplacement EMPL may be disposed on a portion of the body BDY, including bone BNE, tissue TSS, and skin SKN, as well as for disposition on an auxiliary element 157. The open aperture 1510 which is opened in the contact element 151 permits the user to inspect the auxiliary element 157 therethrough, either by visual scrutiny and/or by manual tactile palpation. It is noted that the contact element 151 may be configured as a gimbal mechanism 1511. The user is an operator of the medical apparatus 10. In addition, the shape of the open aperture 1510 may be selected to have a desired geometric shape.

The mechanical mechanism 11 is further configured to support at least one stopper element 20. The at least one stopper element 20 is configured to prevent motion of translation to further close the mechanism 11, thus to bring together of the first portion 12 and the second portion 14 of the mechanical mechanism 11, for example, to prevent deeper drilling of a bore BRE. The stopper element 20 which is supported and coupled to the mechanical mechanism 11, may be controllably disposed and affixed thereon, but releasably so, for adjustment in desired disposition. Thereby, it is possible to adjustably set a gap of separation 210 which separates apart between the contact element 151 and/or the contact point 154 thereof, and a tip 198 of the medical implement 19. Thereby, the at least one stopper element 20 is adjustable to limit a depth of penetration of the medical implement 19 into a blind bore BBOR. The adjustment of disposition of the at least one stopper element 20 may be obtained by either or both of fine adjustment control and coarse adjustment control.

The contact point 154 of the contact element 151 may further be configured for disposition on an auxiliary element 157 which is supported on the body BDY. However, care is taken, for example by tactile palpation and/or by visual inspection, to maintain the contact point 154 in disposition along the straight segment of line which is aimed at the selected emplacement EMPL.

The first portion 12 and the second portion 14 of the mechanical mechanism 11 are configured to operate in a first plane of operation 1PL. A first additional portion 128 is coupled to the first portion 12 which supports the contact module 15, and a second additional portion 148 is coupled to the second portion 14 which supports the operation module 16. Both the first additional portion 128 and the second additional portion 148 are configured to operate in a second plane of operation 2PL. However, the first plane of operation 1PL and the second plane of operation 2PL may be either one out of the same plane and a different plane. Nevertheless, the medical apparatus 10 is further configured to remain an independent handheld apparatus of rigid mechanical structure. Furthermore, the medical implement 19 remains permanently aimed at the open aperture 1510 along the segment of straight line, irrelevantly of one out of motion and standstill of the mechanical mechanism 11 and at least one out of the medical implements 19. Moreover, the implement guide 17 is further configured to support at least one medical implement 19 in aim at the contact point of the contact element 151, and the implement drive 18 is configured to drive the at least one medical implement 19 alone and in combination, in at least one out of linear translation motion, rotational motion, and reciprocation motion.

In another exemplary embodiment, the medical apparatus 10 may be configured as a structure medical apparatus 310 having the mechanical structure 311 for guiding and orienting a medical implement 19. The structure medical apparatus 310 may support a first structure portion 312, a second structure portion 314, and a third structure portion 313, the three of which are rigidly coupled together. The structure medical apparatus 310 may have the curved shape of the letter C wherein one end portion of the letter C supports the contact unit 315 and the other end portion of the letter C supports the structural implement guide 171.

The first structure portion 312 may support a contact unit 315 having a contact element 3151 configured for disposition on a selected emplacement EMPL on a portion of a body BDY, and the second structure portion 314 may support a structural implement guide 171 which holds and guides a medical implement 19. In addition, the mechanical structure 311 is configured to permanently guide and aim a longitudinal axis 199 of the medical implement 19 at the contact element 3151. Thereby, the mechanical structure 311 is configured to maintain the medical implement 19 in aim at the contact element 3151 in coaxial alignment relative to each other along a straight segment of a line.

The structural implement guide 171 may be configured to support at least one dental implement 19 and at least one adaptor sleeve 173. The contact element 3151 may have an open aperture 3158 which is configured for inspection therethrough by at least one out of visual scrutiny and tactile palpation of the selected emplacement EMPL which is disposed on a portion of the body BDY, including bone BNE, tissue TSS, and skin SKN.

The contact point 3154 of the contact element 3151 may further be configured for disposition on an auxiliary element 3157 which may be supported on the body BDY and is oriented along the straight segment of line. However, care is taken, for example by tactile palpation and/or by visual inspection, to maintain the contact point 3154 in disposition along the straight segment of line which is aimed at the selected emplacement EMPL.

The structure medical apparatus 310 may be configured as an independent handheld apparatus of rigid mechanical structure 311 may be made by additive printing process.

The contact element 3151 may be configured to support a gimbal mechanism 1511.

The structural implement guide 171 is further configured to operate in association with a handheld motorized drive 181 to which a medical implement 19 is coupled. In addition, the medical implement 19 may be driven by the motorized drive 181, alone and in combination, in at least one out of linear translation motion, rotational motion, and reciprocal motion.

The medical apparatus 10 and the structure medical apparatus 310 are appropriate for production by the medical apparatus and medical tools industries.

Use of the medical apparatus 10 or structure medical apparatus 310 can ensure that by mechanically guiding medical implements 19, the medical implements 19 may be aimed from an entry point NTRPNT to a selected target point TRGPNT and will not deviate away from the straight segment of straight line stretching from the entry point NTRPNT to the target point TRGPNT. Thereby, damage to the patient, sometimes irreparable damage, is avoided.

Likewise, the stopper element 20, or stopper 20, can limit the depth of a blind bore BBOR, and to prevent drilling farther or deeper than desired, meaning past and away from the target point TRGPNT. Thereby, in the case of blind bores, such as for implants, physical damage to the body BDY is prevented, which damage is sometimes irreversible.

The medical apparatus 10 and structure medical apparatus 310 are independent and do not rely on exterior equipment by being configured to operate. For example, assistance from imaging facilities and/or computer processors operating dedicated computer programs is not required.

Simplicity of the mechanical mechanism, of the mechanical structure, and of use are further outstanding advantages of the medical apparatus 10, in addition to being lightweight, rigid and robust, and being produced from medical practice compliant materials. Such materials may include metals, plastics, and synthetic materials. Moreover, fabrication of the medical apparatus 10 may be achieved by conventional manufacturing processes.

1. A medical apparatus for guiding a medical implement, the medical apparatus comprising:
a mechanical mechanism comprising:
a first mechanism portion coupled to a first additional portion, and including a contact element or contact point that is configured for disposition on a selected emplacement on a portion of a body, and
a second mechanism portion coupled to a second additional portion, and including an operation module configured for guidance and support of the medical implement,
wherein the mechanical mechanism is configured to:
permit displacement of the contact element and the operation module relative to each other in bidirectional translational motion along a segment of a straight line, and
maintain the medical implement in permanent aim at the selected emplacement which is selected by disposition of the contact element or contact point,
wherein the first portion and the second portion are disposed in a first plane of operation, and the first additional portion and the second additional portion of addition are disposed in a second plane of operation, and
wherein the first plane of operation and the second plane of operation are either the same plane or different planes.

2. The apparatus of claim 1, wherein the mechanical mechanism is configured to aim the medical implement at the contact element.

3. The apparatus of claim 1, wherein the operation module is configured to support a plurality of different medical implements.

4. The apparatus of claim 1, wherein the operation module is configured to support at least one of an implement guide, and an implement guide associated with an implement drive.

5. The apparatus of claim 4, wherein the implement guide supports at least one of a guide sleeve, and a guide sleeve which supports an adaptor sleeve.

6. The apparatus of claim 4, wherein the operation module supports the implement guide associated with the implement drive, and the implement drive is configured to both support a motorized drive to which a medical tool is coupled, and to drive the medical implement into motion.

7. The apparatus of claim 6, wherein the motorized drive is a hand-held power tool.

8. The apparatus of claim 1, wherein the contact element is configured for selective disposition on bone, tissue, or skin, as the selected emplacement, and the contact element is further configured for disposition on an auxiliary element coupled to the body.

9. The apparatus of claim 1, wherein the contact element comprises an open aperture which is configured to enable visual inspection and/or tactile palpation therethrough of the selected emplacement.

10. The apparatus of claim 1, wherein the mechanical mechanism further comprises at least one stopper element which is adjustable to set a gap of separation between the contact element and a tip of the medical implement.

11. The apparatus of claim 10, wherein the stopper element is adjustable by at least one of a fine adjustment control and a coarse adjustment control.

12. The apparatus of claim 1, wherein:
the contact element is disposed on an auxiliary element which is configured to be supported on the body, and
the contact element is configured such that when the auxiliary element is supported on the body with the contact element disposed on the selected emplacement, the contact element is disposed on a straight line segment which is aimed at the selected emplacement from the medical implement.

13. The apparatus of claim 1, wherein the first plane of operation and the second plane of operation are different planes and are disposed at an angle α relative to each other, wherein the angle α is fixed or adjustable.

14. The apparatus of claim 1, which is configured as an independent handheld apparatus of rigid mechanical structure.

15. The apparatus of claim 9, wherein the contact element comprises a gimbal mechanism having at least three degrees of freedom of rotation.

16. The apparatus of claim 6, wherein in operation, the motorized drive and the implement guide are maintained jointly together by manual retention.

17. The apparatus of claim 9, wherein the medical implement is permanently aimed at the open aperture along the segment of straight line, regardless of motion of the mechanical mechanism and motion of the medical implement.

18. The apparatus of claim 4, wherein:
the operation module supports the implement guide associated with the implement drive,
the implement guide is configured to support the medical implement in aim at the contact element, and
the implement drive is configured to drive the at medical implement in at least one of linear translation motion, rotational motion, and reciprocation motion.

19. The apparatus of claim 10, wherein the at least one stopper element is adjustable to limit a depth of penetration of the medical implement into a blind bore.

20. The apparatus of claim 9, wherein the open aperture has a shape that allows a human fingertip to pass therethrough to make manual tactile contact with the selected emplacement.

21. A method for constructing a medical apparatus for guiding a medical implement for implantation of medical implants, including at least one of orthopedic implants, dental implants and zygomatic implants,
the method comprising:
constructing a mechanical mechanism by providing a first mechanism portion coupled to a first additional portion and having a contact element configured for disposition on a selected emplacement on a portion of a body, and providing a second mechanism portion coupled to a second additional portion of addition and having an operation module configured for supporting a medical implement, such that the first portion and the second portion are disposed in a first plane of operation, and the first additional portion and the second additional portion are disposed in a second plane of operation, the first plane of operation and the second plane of operation being either the same plane or different planes, and
adapting the mechanical mechanism for aiming a longitudinal axis of the medical implement at the open aperture of the contact element, and for providing mutual relative displacement between the operation module and the contact element in bidirectional translation motion along a segment of a straight line and in permanent mutual aim of the medical implement at the contact element in an orientation directed along the straight segment of line,
wherein the contact element comprises a gimbal mechanism having at least three degrees of freedom of rotation.

22. The method of claim 21, wherein the operation module is configured to support a plurality of different medical implements.

23. The method of claim 21, wherein:
the operation module includes an implement guide, and an implement drive which is slidable into and out of the implement guide, and
retrieval of the implement drive out of the implement guide allows the implement guide to support and guide medical implements therein.

24. The method of claim 23, wherein the implement guide is configured to support and guide various dental implements by use of diameter-matching adaptor sleeves.

25. The method of claim 24, wherein the adaptor sleeves are supported in the implement guide in exchangeable engagement therein and releasable extraction thereout.

26. The method of claim 23, wherein:
the implement drive is configured to support a handheld motorized drive configured for providing motion to a dental implement supported thereby, and
the handheld motorized drive is a handpiece.

27. The method of claim 21, wherein the contact element comprises an open aperture which is configured to enable visual inspection and/or tactile palpation therethrough of the selected emplacement.

28. The method of claim 21, wherein the gimbal mechanism comprises a finger rest which is releasable and interchangeable.

29. The method of claim 23, wherein:
the implement guide is configured to support the medical implement in aim at the contact element, and
the implement drive is configured to drive the medical implement in at least one of linear translation motion, rotational motion, and reciprocation motion.

30. A medical apparatus for guiding a motor-driven medical implement 19, the medical apparatus comprising:
a mechanical mechanism supporting:
a first portion coupled to a first additional portion of which supports a contact element configured for disposition on a selected emplacement on a portion of a body, and
a second portion coupled to a second additional portion which supports, holds, and guides the medical implement,
wherein the first portion and the second portion are disposed in a first plane of operation, and the first additional portion and the second additional portion are disposed in a second plane of operation, the first plane of operation and the second plane of operation being either the same plane or different planes, and
wherein the mechanism is configured to permanently guide and aim the medical implement at the contact element and to permit displacement of the contact element and of the medical implement relative to each other in bidirectional motion of translation along a segment of a straight line.

31. The apparatus of claim 30, wherein:
the mechanical mechanism is configured to maintain the medical implement in aim at the contact element in coaxial alignment relative to each other along a straight segment of a line, and
the contact element comprises a digit window for a fingertip to touch the emplacement.

32. The apparatus of claim 30, wherein:
the first plane and the second plane are different planes and are disposed at an angle α relative to each other, and
the angle α is fixed or adjustable.

33. The apparatus of claim 30, wherein the contact element comprises an open aperture which is configured to enable visual inspection and/or tactile palpation therethrough of the selected emplacement.

34. The apparatus of claim 31, wherein the contact element is disposed on an auxiliary element which is configured to be held in contact with the body.

35. The apparatus of claim 30, wherein the contact element comprises a gimbal mechanism.

36. The apparatus of claim 30, wherein the contact element comprises a gimbal mechanism having at least three degrees of freedom of rotation.

37. The apparatus of claim 30, wherein the motor-driven medical implement is configured to be driven by a handheld motorized drive in at least one of linear translation motion, rotational motion, and reciprocation motion.
